(19) United States
(12) Patent Application Publication
(10) Pub. No.: US 2026/0061138 A1
(43) Pub. Date: Mar. 5, 2026

(54) GRIPPER

(71) Applicant: Novartis AG, Basel (CH)

(72) Inventors: Neil Cammish, Manchester (GB); Mark Horlock, Cheshire (GB); Christophe Raossanaly, Basil (CH)

(21) Appl. No.: 19/382,581

(22) Filed: Nov. 7, 2025

Related U.S. Application Data
(63) Continuation of application No. PCT/IB2024/054495, filed on May 8, 2024.
(60) Provisional application No. 63/501,281, filed on May 10, 2023.

(51) Int. Cl. A61M 5/32

(52) U.S. Cl. CPC A61M 5/3204

(57) ABSTRACT

The invention relates to a gripper comprising a gripper body. The gripper body extends around, and defines, an opening through which a first axis extends. The opening lies in an opening plane. The gripper further comprises a plurality of teeth and each tooth extends towards the first axis along a tooth axis. Each tooth comprises a root portion and a body portion. The body portion comprises a planar tip portion and the root portion connects the tooth to the gripper body and defines a region about which the tooth can resiliently bend relative to the body. The planar tip portion is resiliently biased to an initial position in which the planar tip portion of each tooth lies in a plane which is offset from the opening plane along the first axis.

### RELATED APPLICATIONS

This application claims the benefit of and priority to U.S. Provisional Application No. 63/501,281, filed May 10, 2023, the entire contents of which are hereby incorporated by reference in their entirety.

### BACKGROUND

The present invention relates to a gripper, particularly to a gripper for removing a needle shield from a needle around which it is coupled, to an injection device including such a gripper, and also to a method of using such a gripper to remove a needle shield.

Medicament delivery devices have been developed to allow a user to self-administer medicament via injection. The injection needles of many of these injection devices are provided with some form of removable needle cover assembly which includes a needle shield, also known as a Rigid Needle Cover or Rigid Needle Shield (RNS).

These needle shields cover the needle to prevent needle stick injuries, and to maintain sterility of the needle, before use. Many examples of needle shields are pushed onto the needle and frictionally engage with a hub of the needle or delivery device which retains the needle shield in place covering the needle. An inner portion of the needle shield may be made of a resiliently deformable material such as a rubber or thermoplastic elastomer (TPE). This can help to provide and maintain a good seal between the needle shield and the delivery device. However, providing a reliable seal can present problems for users in removing the needle shield prior to use, particularly for users with impaired manual dexterity.

Devices have been developed which can receive or house a syringe or cartridge with an attached needle which is protected by a rigid needle shield. These devices may include a removable cap which includes a gripping device or mechanism therein to engage and pull the needle shield from the needle when the cap is removed in order to make the device ready to use. There are many different designs of needle shield and gripping devices may be specific to a particular design.

### SUMMARY

The present invention provides a gripper comprising a gripper body, the gripper body extending around, and defining, an opening through which a first axis extends, the opening lying in an opening plane, the gripper further comprising a plurality of teeth, each tooth extending towards the first axis along a tooth axis, each tooth comprises a root portion and a body portion, the body portion comprising a planar tip portion, the root portion connects the tooth to the gripper body and defines a region about which the tooth can resiliently bend relative to the body, the planar tip portion being resiliently biased to an initial position in which the planar tip portion of each tooth lies in a plane which is offset from the opening plane along the first axis.

The gripper is particularly intended for use in an injector device, to remove a needle shield from a needle around which it is coupled, but the gripper could be used to grip other objects. As an example, during use of the gripper to grip a needle shield, a needle shield can be inserted though the opening along the first axis. To insert a needle shield through the opening of the needle shield the needle shield is moved relative to the gripper along the first axis. This movement will cause an outer surface of the needle shield to contacts the teeth. With the teeth in the initial position the needle shield may not be able to pass between the teeth. The teeth terminate away from the first axis and define an inner opening. It should be noted that movement along the first axis may be parallel with the first axis. The planar tip portion of each tooth may lie in a plane which is substantially parallel with the opening plane.

The gripper may be arranged such that, in use, the needle shield may pass through the opening prior to contacting the teeth as this arrangement can provide greater flexibility in the positioning of the gripper in a device, for example in a device cap. A skilled person will understand that it would be possible to arrange the gripper such that the needle shield contacts the teeth prior to passing through the opening, but this arrangement may provide less flexibility in positioning.

The offsetting of the offset plane and the opening plane may be at least 0.5 mm, 1 mm, 1.5 mm or at least 2 mm. A maximum dimension of the body, for example, a radius of a circular body, may be at least 10 mm, 12 mm, 15 mm, or at least 17 mm and may be less than 20 mm.

An insertion force causes further relative movement of the gripper and needle shield along the first axis in a first direction and this causes the teeth to bend at the root away from their initial position. The bending of the teeth at the root causes the planar tip portions to move apart such that the needle shield can pass between the planar tip portions. If the needle shield has passed through the opening prior to contacting the teeth, the planar tip portions will also move away from the opening. The teeth moving apart increase the size of the inner opening so that the needle shield can pass between the teeth. The resilient biasing of the teeth towards the initial position causes the teeth to remain in contact with the outer surface of the needle shield and this inhibits withdrawal of the needle shield from the gripper in a direction opposite the first direction.

The offsetting of the planar tip portions from the opening increases the distance between the point of contact between the needle shield and planar tip portions and the root about which the tooth is intended to resiliently bend which reduces the force required to insert the needle shield through the gripper. The angles at which the forces act are also altered to facilitate insertion of the needle shield.

The part of each tooth in contact with the needle shield may be a contact end of the tooth. The resistance to, or inhibition of, the withdrawal of the needle shield may be due to friction between the contact end and the needle shield alone. In some examples the contact end of each tooth may be shaped, for example sharpened or including projections, such that the contact end digs into an outer surface of the needle shield to provide or create a mechanical feature which inhibits withdrawal of the needle shield from the gripper in a direction opposite the first direction.

The offsetting of the planar tip portions of the teeth from the opening plane alters the force required to insert a needle shield into the gripper and the force with which the teeth can resist withdrawal of the needle shield. The planar tip portions may be offset from the opening plane in the first direction such that, during insertion, the needle shield passes through the opening plane prior to contacting the teeth as this may reduce the insertion force and increase the force which resists withdrawal.

A reduction of insertion force may facilitate assembly of a device which includes a gripper, while an increase in the withdrawal resistance force reduces the risk that a needle shield may be accidentally withdrawn from the gripper during use.

The gripper may be included in a delivery device. The gripper may be secured in a cap which is to be removed from the delivery device body prior to use of the delivery device. The cap may be secured in the cap such that it is movable, for example it may be slidable and/or rotatable relative to the cap. The device may be freely rotatable relative to the cap, or rotatable through only a certain angular range. The device may slide relative to the cap within a predetermined linear range. The device may be, for example, an injector device, such as an autoinjector, which includes a syringe or cartridge to which a needle protected by a needle shield may be connected. The needle may be integrally formed with, or may be releasably connected to, the syringe or cartridge.

The syringe or cartridge may be arranged in the delivery device body such that during assembly of the delivery device the needle shield is inserted into, and is retained in, the gripper. If the gripper is secured in a cap of the delivery device, retaining the needle shield in the gripper means that when the cap is removed prior to use of the delivery device the needle shield will also be removed thereby exposing the needle for use.

The gripper body may be any suitable shape. The gripper body may have a regular outer shape, for example circular or polygonal, or may be an irregular shape. A regular shape, and particularly a circular body or a polygonal shape with more than six sides, may facilitate rotation of the gripper relative to a cap and this may help to reduce the risk of coring of a safety shield by the needle.

The gripper body defines an opening which lies in an opening plane. The gripper body may extend entirely around a periphery of the opening, or the body may include a split or break so that the body surrounds a majority, but not all, of the opening. The shape of an outer periphery of the body may substantially match the shape of an outer periphery of the opening, for example they may both be circular.

The opening lies in the opening plane and at least a portion of the gripper body may also lie in the opening plane. The gripper body may be substantially planar as a substantially flat portion the body may simplify manufacture. The gripper body may include an outer peripheral wall extending at an angle therefrom and this may enhance the bending resistance of the body. The outer peripheral wall may extend substantially perpendicular to the gripper body. The outer peripheral wall may extend along the first axis and may be formed on one side of the body only and this may be the same side of the body to which the planer tip portions of the teeth are offset. The outer peripheral wall may extend substantially parallel with the first axis. The outer peripheral wall may extend entirely around the gripper body.

The first axis extends though the opening and the first axis may be substantially perpendicular to the opening plane. The first axis may be substantially central in the opening.

The teeth may extend from a root portion towards the first axis. The root portion connects the tooth to the body. At least some of the root portion may lie in the opening plane. The root portion may be located on an inner periphery of the body which defines the opening as this may simplify manufacture of the gripper.

The body portion of the tooth extends away from the root portion and includes a planar tip portion. The body portion of each tooth may have any suitable shape and may include ridges, steps or other features which may alter the stiffness of the body portion. The bodies of each of the plurality of teeth may be the same shape.

In use, the body portions of the teeth may be contacted by a needle shield being inserted through the opening through the gripper body. The insertion force will cause the teeth to be forced away from their initial positions by bending at their root and thereby cause the planar tip portions to move apart such that the needle shield can pass between them. The root is resiliently deformable so that the teeth are resiliently biased to an initial position.

The root portion of each tooth may be waisted, or narrowed, to facilitate bending of the tooth about the root. Wasting of the tooth is a narrowing of the material of the tooth to create a narrowed waist area. The narrowing may be in the thickness of the material along the first axis, or may be in a direction perpendicular to the first axis and the tooth axis such that a width of the tooth is narrowed in the root portion. Narrowing in in a direction perpendicular to the first axis and the tooth axis such that a width of the tooth is narrowed in the root portion may simplify manufacturing of the gripper. Each tooth may be widest in a direction perpendicular to the first axis and the tooth axis between the root portion and an end of the tooth.

A width of the tooth in a direction perpendicular to the first axis and the tooth axis may reduce towards the first axis along at least a portion of the tooth body. For example the width may taper along the tooth axis from the root towards a contact end.

The gripper body may be annular, or a polygon with a hole therethrough, for example a polygon with more than six sides. The shape of the hole therethrough may match the external shape of the body.

The gripper body may further comprise a peripheral wall around, and extending from, an outer perimeter of body. The peripheral wall may extending away from the body along the first axis, or at any non-zero angle from the gripper body as this may enhance the bending resistance of the gripper body. The peripheral wall may extend substantially parallel with the first axis. The peripheral wall may extend substantially entirely around the gripper body.

The body portion of each tooth may comprise an intermediate portion between the root and the tip portion. The intermediate portion may be substantially planar and offset from the opening plane along the first axis. This may result in an intermediate portion arranged such that, when the planer tip is in the initial position, the intermediate portion is located between the opening plane and the tip portion.

Each tooth may comprise at least one step which offsets the tip portion from the root portion along the first axis. The step is a discontinuity in the shape of the tooth rather than a smooth curve. For example a portion of the step may extend at an angle of at least 45° to the opening plane, at least 55° to the opening plane, at least 65° to the opening plane, at least 75° to the opening plane, or substantially parallel with the first axis.

Each tooth may comprise a first step which offsets the intermediate portion from the root portion along the first axis, and may comprise a second step which offsets the tip portion from the intermediate portion along the first axis. The, or each, step may include a portion that is substantially parallel with the first axis when the planer tip is in the initial position.

There may be any suitable number of teeth, for example three teeth, four teeth, five teeth or six teeth. The plurality of teeth may be substantially equally distributed around the opening as this may reduce mis-matched forces being applied to the needle shield.

The planar tip portion may comprise a contact end which, in use, will contact an outer surface of a needle shield. The contact end may being curved or faceted around the first axis such that it is a substantially constant distance therefrom.

The gripper may be manufactured by any suitable method, for example moulding, or forming, and may be manufactured from any suitable material, for example metal, plastic, or a combination of materials. The gripper may be made of a metal and may be fabricated by stamping the gripper from a metal sheet. The griper may be manufactured as single unitary component.

The body portion of each tooth may include a stiffening ridge to enhance the resistance to bending of the body portion. The stiffening ridge may not extend into the root portion, at least not into the waisted portion of the root, as it is intended that the tooth will bend in the root portion.

The invention further includes a method of removing a needle shield from around a needle using a gripper, the gripper being as claimed in any preceding claim, the method comprising inserting a portion of the needle and needle shield though the opening of the gripper such that the needle shield contacts the plurality of teeth and forces them away from the initial position, continuing to insert the needle and needle shield though the opening of the gripper such that a contact end of the tip portions of the teeth engages side walls of the needle shield, then withdrawing the needle from the opening, the teeth exerting a retaining force on the needle shield to separate the needle shield from the needle as it is withdrawn.

The gripper may be installed in a part of an injection device, for example in a cap of an autoinjector.

### BRIEF DESCRIPTION OF THE FIGURES

The invention will now be described by way of example only with reference to the following figures in which:

FIGS. 1 and 2 show a schematic cross section through a gripper and a plan view of the gripper respectively;

FIGS. 3, 4, and 5 show a side view sequence of a gripper in use;

FIG. 6 shows a schematic cross section through a gripper which includes a side wall and an intermediate portion;

FIG. 7 shows a schematic cross section through a gripper which includes a side wall and an intermediate portion and shaped contact portions;

FIGS. 8 and 9 show a plan and a perspective view of a gripper which includes a side wall;

FIGS. 10 and 11 show a plan and a perspective view of a gripper with an intermediate portion and a side wall;

FIGS. 12 and 13 show a plan and a perspective view of a gripper with a stiffening ridge; and

FIGS. 14 and 15 show views a gripper in use in an injector device.

### DETAILED DESCRIPTION

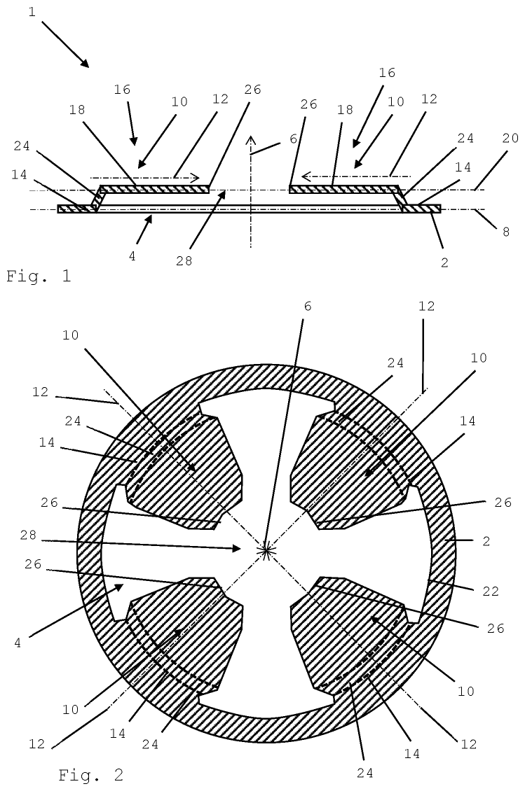

FIG. 1 shows a schematic cross section through a gripper 1 and FIG. 2 shows a plan view of the gripper 1 of FIG. 1. The gripper 1 comprises a gripper body 2 which extends around, and defines, an opening 4 through which a first axis 6 extends. The opening 4 lies in an opening plane 8.

The gripper 1 further comprises a plurality of teeth 10. Each tooth 10 extends towards the first axis 6 along a tooth axis 12. Each tooth comprises a root portion 14 and a body portion 16, and the body portion comprises a planar tip portion 18.

The root portion 14 connects the tooth to the gripper body 2 and comprises a waited region about which the tooth 10 can resiliently bend relative to the gripper body 2. The root portion 14 may lie in the opening plane 8 as in this example. The planar tip portion 18 is resiliently biased to an initial position in which the planar tip portion 18 of each tooth lies in an offset plane 20 which is offset from the opening plane 8 along the first axis 6.

The gripper body 2 entirely surrounds the opening 4. The opening 4 is defined by an inner periphery 22 of the gripper body 2. The teeth 10 extend from the inner periphery 22 of the gripper body 2 towards the first axis 6. A root portion 14 couples each tooth body 16 to the inner periphery 22 of the gripper body 2.

The root portion 14 includes a waisted, or narrowed region, adjacent the inner periphery 22 of the gripper body 2. The root portion 14 also includes a root step 24 which extends along the first axis 6 to offset the planar tip portion 18 from the opening plane 8.

The teeth 10 each extend along a tooth axis 12 towards the first axis 6. A contact surface 26 at a distal end of each planar tip portion 18, but is set back from the first axis 6 to define an inner opening 28 around the first axis 6. The contact surface 25 of each tooth 10 is curved, or comprises a plurality of lines, such that the contact surface 26 remains at a substantially constant distance from the first axis 6.

FIGS. 3, 4, and 5 show a side view sequence of a gripper 101 in use. The gripper 101 is similar to the gripper 1 of FIG. 1 and similar features will be references with similar numbers incremented by 100. The gripper 101 comprises a body 102 which defines an opening 104 and teeth 110 which define an inner opening 128. The teeth 110 comprise root portions 114 which connect to the body 102. An insertion direction is shown by arrow 30, which passes through the opening plane 108 and then the offset plane 120.

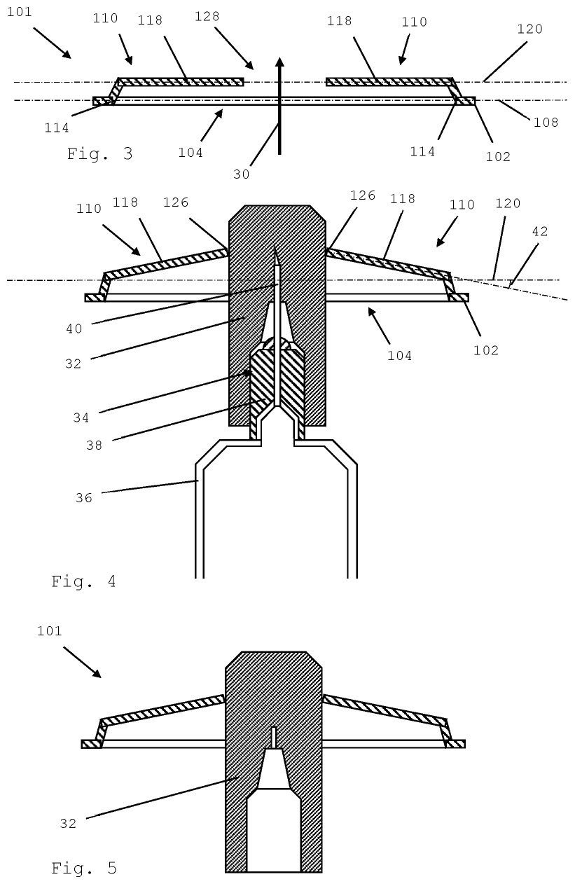

In FIG. 4 the gripper 101 is shown with a needle shield 32 inserted through the opening 104. The needle shield 32 is protecting a needle 34, which is installed on a syringe 36. The needle 34 comprise a needle body 38 and needle tip 40 extending therefrom.

As the needle shield 32 is inserted through the gripper 101 a front end 42 of the needle shield 32 contacts the teeth 110 and, further movement in the insertion direction causes the teeth 110 to deflect through resilient deformation of the root portion 114. This resilient deflection causes the inner opening 128 to increase in size so that it is sufficiently large to accommodate the passage of the needle shield 32 therethrough. As the needle shield 32 passes through the inner opening 128 the contact ends 126 of the teeth 110 are moved away from the opening plane 108 and slide along an outer surface of the needle shield 32.

The insertion of the needle shield 32 through the gripper causes the planar tip portions 118 to be deflected by an angle 42 away from the offset plane 120.

Removal of the needle shield 32 from the gripper 101 is resisted by the interaction of the contact ends 126 and the needle shield 32. Through interaction of contact ends 126 and the needle shield 32, applying a removal force to the needle shield 32, for example by applying a removal force to the syringe 36, would tend to draw the teeth 110 towards their original position. Drawing the teeth 110 back towards their original position reduces the size of the inner opening and so increases the interaction force between the contact ends 126 and the needle shield 32.

As the teeth 110 are angled relative to the offset plane 120 the teeth act to resist removal of the needle shield 32 from the gripper 110.

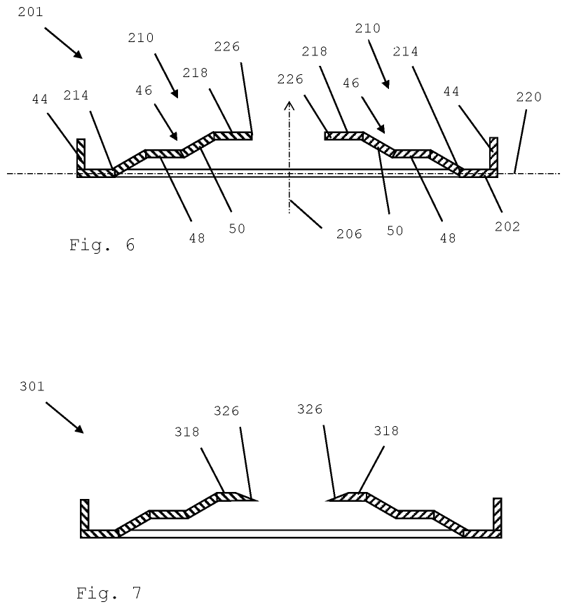

FIG. 5 shows the gripper 101 and needle shield 32 once the syringe 36 and needle 38 have been removed.

FIG. 6 shows a schematic cross section through a gripper 201 which includes a side wall 44 and an intermediate portion 46. The gripper 201 is similar to the gripper 1 of FIG. 1 and similar features will be references with similar numbers incremented by 200.

The gripper 201 includes a side wall 44 which extends from the gripper body 202 in a direction along the first axis 206. The side wall 44 may extend substantially parallel with the first axis 206 and thus stiffen the gripper body 202 against bending.

Each tooth 210 includes an intermediate portion 46 arranged between the root portion 214 and the planar tip portion 218. The intermediate portion 46 may include an intermediate planar portion 48 and an intermediate step 50. The root step 224 provides a first step which offsets the intermediate portion 46 from the opening plane 220 along the first axis 26. The intermediate portion 46 comprises an intermediate planar portion 48 and intermediate step 50. The intermediate step 50 provides a second step which offsets the planar tip portion 218 from the intermediate portion 46 along the first axis.

The intermediate portion 46 is located between the root portion 214 and the planar tip portion 218. The intermediate portion 46 comprises a substantially planar intermediate portion 48 which is offset from the opening plane 220 along the first axis 206 such that, the planer tip portion 218 is in the initial position, the intermediate planar portion 48 is located between the opening plane 220 and the planar tip portions 218.

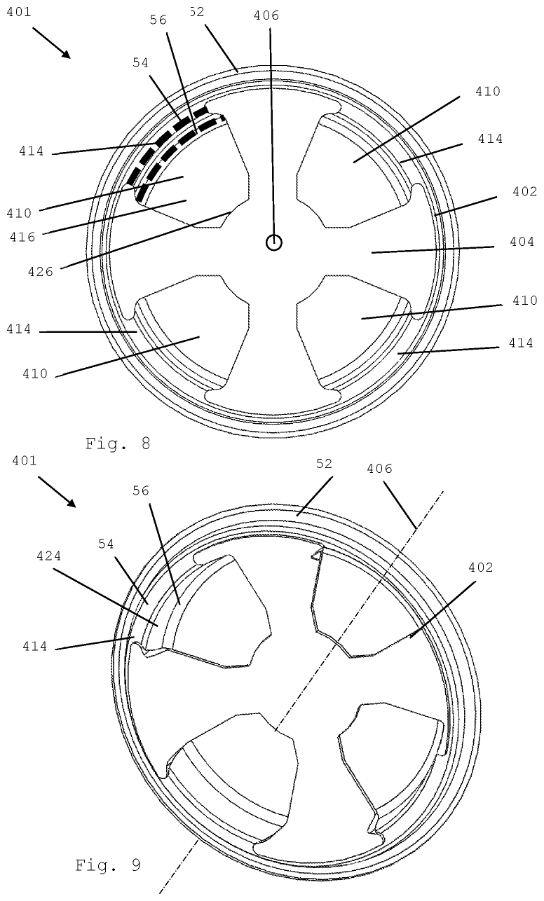

FIG. 7 shows a schematic cross section through a gripper 301. The gripper 301 is similar to the gripper 201 of FIG. 6 and similar features will be references with similar numbers incremented by 100.

The gripper 301 differs from the gripper 201 in that the planar tip portions 318 comprise a shaped contact end 326. In this example the contact end 326 is sharpened so that it will more readily dig into a surface of a needle shield 32 to enhance the engagement between the contact ends 326 and the shield 32. The contact end 326 may be shaped to include sharpened portions, projections or other features to enhance the engagement between the contact ends 326 and the shield 32.

FIGS. 8 and 9 show a plan and a perspective view of a gripper 401. The gripper 401 is similar to the gripper 1 of FIG. 1 and similar features will be referenced with similar numbers incremented by 400.

The gripper 401 comprises an annular body 402 surrounding an opening 404 through which a first axis 406 passes. Teeth 410 extend from the body 402 into the opening towards the first axis 406. A root portion 414 comprises a waisted portion, an example of which is shown as 54 on one tooth 410. The waisted portion 54 is narrower than a portion 56 of the body 416 of the tooth 410 located between the root portion 414 and the contact end 426. The portion 56 of the tooth that is wider than the waisted portion 54 is at the end of root step 424.

The gripper 401 differs from the gripper 1 in that it includes a peripheral side wall 52.

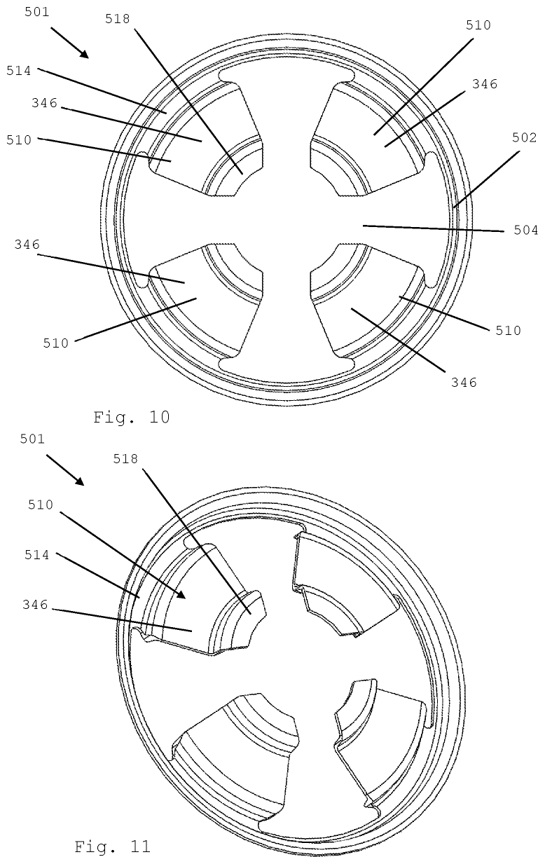

FIGS. 10 and 11 show a plan and a perspective view of a gripper 501. The gripper 501 is similar to the gripper 201 of FIG. 6, and similar features will be referenced with similar numbers incremented by 300.

The gripper 501 comprises an annular body 502 surrounding an opening 504 and differs from the gripper 401 in that each tooth 510 includes an intermediate portion 346 located between the root portion 514 and the planar tip portion 518.

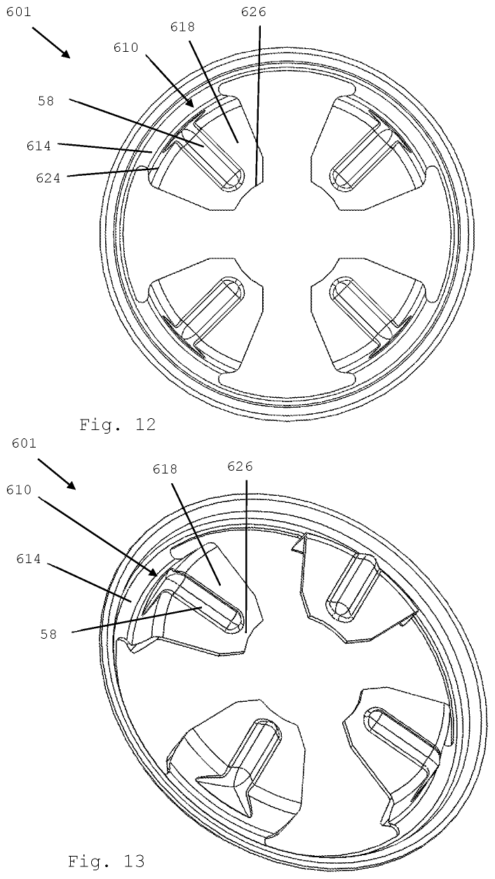

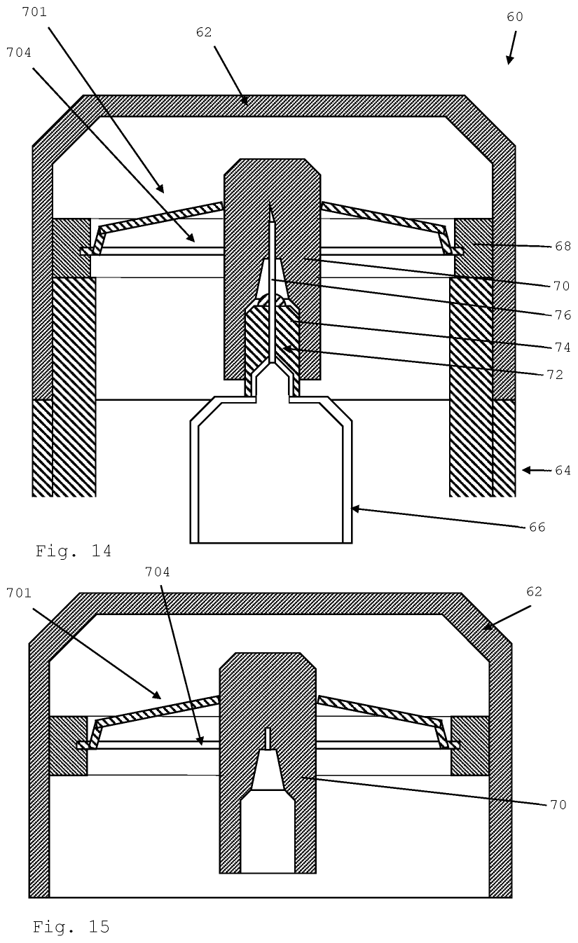

FIGS. 12 and 13 show a plan and a perspective view of a gripper 601. The gripper 601 is similar to the gripper 401 of FIG. 8, and similar features will be referenced with similar numbers incremented by 200.

Each tooth 610 comprises a stiffening ridge 58 which extends along the tooth 610. The stiffening ridge 58 starts on the root step 624 and extends along the planar tip portion 618 and terminates before reaching the contact end 626. The ridge 58 in this example is formed by a deformation which extends away from the opening plane, but could be formed by increasing the thickness of the material, of producing a ridge directed towards the opening plane.

FIGS. 14 and 15 show views a gripper 701 in use in an injector device 60. The injector 60 comprises a cap 62 and a body 64, and the body contains a syringe 66. The cap 62 comprises a connector block 68 to which the gripper 701 is coupled.

The gripper 701 has a needle shield 70 inserted through the opening 704 as the injector 60 has been assembled prior to use. The needle shield 70 is protecting a needle 72, which is installed on the syringe 66. The needle 72 comprise a needle body 72 and needle tip 76 extending therefrom.

In this example, the gripper 701 coupled to the connector block 68 to that the gripper 701 is able to rotate relative to the cap 62 as this reduces the risk of the needle tip coring the needle shield.

As the cap 62 is removed from the body 64 of the injector 60, the syringe 66 is moved away from the cap 62 and, as a result, a removal force is applied to the needle shield 70 to try to remove the needle shield 70 from the gripper 701. The gripper 701 is coupled to the cap 62 and the gripper 701 resists removal of the needle shield 701 from the gripper 701. Thus, removal of the cap 62 from the body results in separation of the needle shield 70 from the needle 72, with the needle shield 70 retained in the cap 62.

1. A gripper comprising a gripper body, the gripper body extending around, and defining, an opening through which a first axis extends, the opening lying in an opening plane, the gripper further comprising a plurality of teeth, each tooth extending towards the first axis along a tooth axis, each tooth comprises a root portion and a body portion, the body portion comprising a planar tip portion, the root portion connects the tooth to the gripper body and defines a region about which the tooth can resiliently bend relative to the body, the planar tip portion being resiliently biased to an initial position in which the planar tip portion of each tooth lies in a plane which is offset from the opening plane along the first axis.

2. The gripper as claimed in claim 1, in which the opening plane is substantially perpendicular to the first axis.

3. The gripper as claimed in claim 1, in which the root portion of each tooth is waisted to facilitate bending of the tooth about the root.

4. The gripper as claimed in claim 3, in which the root portion is waisted in a direction perpendicular to the first axis and the tooth axis.

5. The gripper as claimed in claim 4, in which each tooth is widest in a direction perpendicular to the first axis and the tooth axis between the root portion and an end of the tooth.

6. The gripper as claimed in claim 1, in which the width of the tooth in a direction perpendicular to the first axis and the tooth axis reduces towards the first axis along at least a portion of the tooth body.

7. The gripper as claimed in claim 1, in which the gripper body is annular.

8. The gripper as claimed in claim 7, in which the gripper body further comprises a peripheral wall around, and extending from, an outer perimeter of body, the peripheral wall extending away from the body along the first axis.

9. The gripper as claimed in claim 8, in which the peripheral wall extends parallel with the first axis.

10. The gripper as claimed in claim 1, in which the body portion of each tooth comprises an intermediate portion between the root and the tip portion, the intermediate portion being substantially planar and offset from the opening plane along the first axis such that, when the planer tip is in the initial position, the intermediate portion is located between the opening plane and the tip portion.

11. The gripper as claimed in claim 1, in which each tooth comprises at least one step which offsets the tip portion from the root portion along the first axis.

12. The gripper as claimed in 10, in which each tooth comprises a first step which offsets the intermediate portion from the root portion along the first axis and a second step which offsets the tip portion from the intermediate portion along the first axis.

13. The gripper as claimed in claim 11, in which the, or each, step is substantially parallel with the first axis when the planer tip is in the initial position.

14. The gripper as claimed in claim 1, in which the there are four teeth.

15. The gripper as claimed in claim 1, in which the plurality of teeth are substantially equally distributed around the opening.

16. The gripper as claimed in claim 1 in which the tip portion comprises a contact end, the contact end being curved around the first axis.

17. The gripper as claimed in claim 1, in which the gripper is formed from a metal.

18. The gripper as claimed in claim 1, in which the gripper is formed as unitary structure.

19. The gripper as claimed in claim 1, in which the body portion of each tooth includes a stiffening ridge.

20. A method of removing a needle shield from around a needle using a gripper, the gripper being as claimed in claim 1, the method comprising inserting a portion of the needle and needle shield though the opening of the gripper such that the needle shield contacts the plurality of teeth and forces them away from the initial position, continuing to insert the needle and needle shield though the opening of the gripper such that a contact end of the tip portions of the teeth engages side walls of the needle shield, then withdrawing the needle from the opening, the teeth exerting a retaining force on the needle shield to separate the needle shield from the needle as it is withdrawn.
